(19) United States
(12) Patent Application Publication
(10) Pub. No.: US 2005/0037955 A1
(43) Pub. Date: Feb. 17, 2005

(54) Therapeutic protein and treatments

(76) Inventors: Laura Virginia Hooper, Crestwood, MO (US); Thaddeus Smith Stappenbeck, St. Louis, MO (US); Chieu Vi Hong, St. Louis, MO (US); Jeffrey Ivan Gordon, St. Louis, MO (US)

Correspondence Address: NIXON & VANDERHYE, PC, 1100 N GLEBE ROAD, 8TH FLOOR, ARLINGTON, VA, 22201-4714, US

(21) Appl. No.: 10/496,839

(22) PCT Filed: Nov. 27, 2002
(86) PCT No.: PCT/US02/37711

Related U.S. Application Data
(60) Provisional application No. 60/333,151, filed on Nov. 27, 2001.

(51) Int. Cl. A61K 38/17; C07K 14/47; C07H 21/04

(52) U.S. Cl. 514/12; 435/69.1; 435/320.1; 435/325; 530/350; 536/23.5

(57) ABSTRACT

The present invention relates to microbial (antibacterial and antifungal) and other therapeutic treatments for diseases, in particular but not limited to the intestine, based upon angiogenins, and particularly angiogenin-4: a novel intestine-specific, epithelial-based, microbially-regulated member of the angiogenin family. Novel proteins as well as their use in screening for pharmaceuticals are also described.

The present invention relates to microbial and other therapeutic treatments for diseases, in particular of the intestine, based upon angiogenins, and particularly angiogenin-4: a novel intestine-specific, epithelial-based, microbially-regulated member of the angiogenin family. Novel proteins as well as their use in screening for pharmaceuticals are also described.

### BACKGROUND OF THE INVENTION

The applicants have previously conducted analysis of the transcriptional changes that occur in the intestine as a result of colonization with one of its normal bacterial residents, Bacteroides thetaiotaomicron. The goal of the analysis was to identify and characterize new therapeutic targets that play an important role in normal intestinal function and/or in the pathogenesis of various gastrointestinal diseases. (Hooper, L. V et al. (2001). Science 291:881).

The approach was to use Affymetrix GeneChips to conduct a global assessment of gene expression before and after colonization of germ-free NMRI mice with B. thetaiotaomicron—a prominent, genetically manipulatable human and mouse gut commensal.

### SUMMARY OF THE INVENTION

The applicants have found that a particular member of the angiogenin family is specifically expressed in the intestine, more specifically in the epithelium's Paneth cell lineage. As a result of this, it is believed that this could give rise to a range of therapeutic applications.

A first aspect of the invention provides an isolated polypeptide comprising SEQ ID NO 1 as shown in FIG. 1 hereinafter, or an allelic variant thereof or a polypeptide which has at least 85% amino acid sequence identity with SEQ ID NO 1, or a biologically active fragment of any of these.

The term “polypeptides” refers to peptides including proteins. “Allelic variants” refer to proteins that are encoded by alternative forms of the gene found at the same locus within a mouse genome as the gene which encodes a protein of SEQ ID NO 1.

In a particular embodiment, the invention provides a polypeptide of SEQ ID NO 1 as shown in FIG. 1 hereinafter, or a polypeptide which has at least 85%, suitably at least 90%, more suitably at least 95%, yet more suitably at least 98% and most preferably at least 99% amino acid sequence identity with SEQ ID NO 1 or a or a biologically active fragment of any of these.

Sequence identity can be assessed using any of the known algorithms. For example, identity can be measured using the BLAST P algorithm or the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

As used herein, the term “fragment” refers to any portion of the given amino acid sequence which has a desired biological activity as defined hereinafter. Fragments will suitably comprise at least 10 and preferably at least 50 consecutive amino acids from the basic sequence.

As used herein, the term “biological activity” means that the proteins in this case may be any one of the activities discussed below including antimicrobial or angiogenic activity, which may be tested using any of the conventional methods, including those described below in the examples. Examples of anti-microbial activities include antibacterial and antifungal activities. Angiogenic activity is the ability of proteins to stimulate vasculogenesis, i.e. the development of blood vessels.

In particular, the invention provides a protein of SEQ ID NO 1. This protein has been designated angiogenin-4, and the gene, which encodes it, is Ang4. Ang4 mRNA encodes this protein. SEQ ID NO 1 represents the full length protein whilst SEQ ID NO 44 represents the mature protein and thus SEQ ID NO 44 represents a particularly preferred fragment. SEQ ID NO 44 includes a predicted N-terminal signal peptide sequence. A further particular fragment of the protein of SEQ ID NO 1 is a fragment of SEQ ID NO 44 which lacks the N-terminal signal peptide sequence. The N-terminal signal peptide sequence comprises the first five amino acids, QNERY, of SEQ ID NO 44 and is shown underlined in FIG. 1A.

The protein of SEQ ID NO 1 is a murine protein, which is expressed specifically in mouse intestine. This sequence can be used to design probes and primers aimed at finding homologous proteins from other mammals and those with at least 85% identity form particular embodiments of the invention. Such proteins are referred to hereinafter as analogues.

Analogues may also be identified by perfoming laser capture microdissection (LCM) of Paneth cells, upper crypt epithelium, and villus epithelium from surgical specimens of small intestine of the target species, in particular humans. In particular qRT-PCR assays for human angiogenin and various members of the RNase superfamily known to be present in the human genome and to be expressed in the intestine (these intestinal RNases can be identified from in silico searches of the available databases or by direct analysis of human samples using real time quantitative RT-PCR assays).

In a further aspect, the invention provides a nucleic acid which encodes a polypeptide of the invention as described above. Such nucleic acids may be used in the identification of analogs as described above. In addition, they may be used in the production of polypeptides including proteins of the invention using recombinant DNA technology. For instance, they may be incorporated into plasmids or vectors which may be used to transform cells such as prokaryotic cells such as E. coli, which then express the protein. A particular example of a nucleic acid which encodes SEQ ID NO 1 is shown as SEQ ID NO 5 in FIG. 2, or SEQ ID NO 48 in FIG. 3.

Angiogenin-4 is effective as an antimicrobial agent against certain species of microbes. It seems probable that angiogenin-4 is a component of innate host defense/intestinal epithelial barrier function. It may therefore play a role in the prevention or treatment of enteric infections, or adaptive host responses to inflammatory bowel disease where there is Paneth cell hyperplasia, and so may be used to treat such conditions.

It has been shown that gut commensals, such as Bacteroides thetaiotaomicron regulate angiogenesis in the mouse gut. This regulation depends upon Paneth cells. GeneChip profiling of gene expression in Paneth cells failed to identify any known angiogenic factors. However, Ang4, a member of family of proteins with established angiogenic activity, is specifically expressed in Paneth cells. Moreover, its induction by gut microbes coincides with the induction of angiogenesis (T. S. Stappenbeck et al. P.N.A.S. (2002) 99, 15451-15455).

Thus in a further aspect the invention comprises a method of modifying the angiogenic activity within the intestine of an animal in need thereof, which method comprises affecting the activity of a polypeptide of the invention within the intestine. Functions which may be affected by modifying the levels of angiogenic activity, and particularly the levels of angiogenin-4 protein include nutrient absorption, motility, and growth (including the adaptive response to intestinal resection). It is also possible that an epithelial-based angiogenesis factor could participate in the response to ischemic insults or radiotherapy (GI syndrome), or be involved in the pathogenesis of colorectal neoplasia.

Altering the antimicrobial or angiogenic activity levels can be achieved in various ways. For example, in animals who do not produce angiogenin-4, the activity may be induced by administering a polypeptide of the invention or a mimetic, to the intestine. In particular, the polypeptide administered is angiogenin-4 or an angiogenically active fragment thereof, in particular the mature protein of SEQ ID NO 44, which optionally lacks the N-terminal signal sequence.

Where angiogenin-4 is produced, the activity may be enhanced by administration of a polypeptide of the invention, or an agonist or mimetic thereof into the intestine.

This may be achieved for example by formulating the polypeptide of the invention, agonist or mimetic into a formulation which is delivered to the intestine. For example, the formulation may be coated with an enteric coating which is resistant to gastric acid, but which dissolves in the more alkaline environment found in the intestine, to release the protein there.

In addition, the expression of angiogenin-4 is known to be regulated by components of the microbiota (including B. thetaiotaomicron) and therefore administration of such organisms, or chemical entities identified as being produced by components of the microbiota, such as microbial lipopolysaccharides (LPS) may be effective in raising angiogenin-4 levels.

However, where angiogenin-4 is produced, the activity level may also be reduced in order to achieve the desired therapeutic effect. Reduction of the levels of angiogenin-4 in the intestine can be achieved by administering an antagonist of protein activity, or a compound or antibody, which blocks the activity of the protein by binding to it and inactivating it. Reduction of the levels of angiogenic activity may be desirable, for example in the treatment of intestinal cancers, or for treatments of conditions in which it is desirable to suppress nutrient absorption, motility, or growth or other functions as listed above.

Knowledge of the gene according to the invention also provides the ability to regulate its expression in vivo by for example the use of antisense DNA or RNA. One therapeutic means of inhibiting or dampening the expression levels of a particular gene (for example Ang4 identified herein) is to use antisense therapy. Antisense therapy utilises antisense nucleic acid molecules that are synthetic segments of DNA or RNA (“oligonuclotides”), designed to mirror specific mRNA sequences and block protein production. Once formed, the mRNA binds to a ribosome, the cell's protein production “factory” which effectively reads the RNA sequence and manufactures the specific protein molecule dictated by the gene. If an antisense molecule is delivered to the cell (for example as native oligonucleotide or via a suitable antisense expression vector), it binds to the messenger RNA because its sequence is designed to be a complement of the target sequence of bases. Once the two strands bind, the mRNA can no longer dictate the manufacture of the encoded protein by the ribosome and is rapidly broken down by the cell's enzymes, thereby freeing the antisense oligonucleotide to seek and disable another identical messenger strand of mRNA.

With knowledge of the Ang4 gene and mRNA sequence taught herein, the person skilled in the art is able to design suitable antisense nucleic acid therapeutic molecules and administer them as required.

Antisense oligonucleotide molecules with therapeutic potential can be determined experimentally using well established techniques. To enable methods of down-regulating expression of the Ang4 gene of the present invention in mammalian cells, an example antisense expression construct can be readily constructed for instance-using the pREP10 vector (Invitrogen Corporation). Transcripts are expected to inhibit translation of the gene in cells transfected with this type of construct. Antisense transcripts are effective for inhibiting translation of the native gene transcript, and capable of inducing the effects (e.g., regulation of tissue physiology) herein described. Oligonucleotides which are complementary to and hybridisable with any portion of Ang4 gene mRNA are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from the Ang4 gene sequence are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

As noted above, antisense nucleic acid molecules may also be provided as RNAs, as some stable forms of RNA are now known in the art with a long half-life that may be administered directly, without the use of a vector. In addition, DNA constructs may be delivered to cells by liposomes, receptor mediated transfection and other methods known to the art.

The antisense DNA or RNA for co-operation with the target gene can be produced using conventional means, by standard molecular biology and/or by chemical synthesis as described above. If desired, the antisense DNA or antisense RNA may be chemically modified so as to prevent degradation in vivo or to facilitate passage through a cell membrane and/or a substance capable of inactivating mRNA, for example ribozyme, may be linked thereto and the invention extends to such constructs. The antisense DNA or antisense RNA may be of use in the treatment of diseases or disorders in humans in which the over- or under-regulated production of the Ang4 gene product has been implicated.

Alternatively, ribozyme molecules may be designed to cleave and destroy the Ang4 mRNA in vivo. Ribozymes are RNA molecules that possess highly specific endoribonuclease activity. Hammerhead ribozymes comprise a hybridising region, which is complementary in nucleotide sequence to at least part of the target RNA, and a catalytic region, which is adapted to recognise and cleave the target RNA. The hybridising region preferably contains at least 9 nucleotides. The design, construction and use of such ribozymes is well known in the art and is more fully described in Haselhoff and Gerlach, (Nature. 334:585-591, 1988). In another alternative oligonucleotides designed to hybridise to the 5′-region of the Ang4 gene so as to form triple helix structures may be used to block or reduce transcription of the Ang4 gene. In another alternative, RNA interference (RNAi) oligonucleotides or short (18-25 bp) RNAi Ang4 sequences cloned into plasmid vectors are designed to introduce double stranded RNA into mammalian cells to inhibit and/or result in the degradation of Ang4 messenger RNA. Ang4 RNAi molecules may begin adenine/adenine (AA) or at least (any base-A,U,C or G)A . . . . and may comprise of 18 or 19 or 20 or 21 or 22 or 23, or 24 or 25 base pair double stranded RNA molecules with the preferred length being 21 base pairs and be specific to individual Ang4 sequences with 2 nucleotide 3′ overhangs or hairpin forming 45-50mer RNA molecules. The design, construction and use of such small inhibitory RNA molecules is well known in the art and is more fully described in the following: Elbashir et al., (Nature. 411(6836):494-498, 2001); Elbashir et al., (Genes & Dev. 15:188-200, 2001); Harborth, J. et al. (J. Cell Science 114:4557-4565, 2001); Masters et al. (Proc. Natl. Acad. Sci. USA 98:8012-8017, 2001); and, Tuschl et al., (Genes & Dev. 13:3191-3197, 1999).

Identification of agonists or antagonists or blocking compounds may be carried out using standard methods.

Agonists or antagonists may be identified for example by a suitably designed functional assay. Test compounds, for instance from combinatorial libraries, or other compound, or peptide collections, may be administered to cells that express the protein. These cells may be assayed for the biological activity indicative of the presence of angiogenin-4 (e.g. antimicrobial or angiogenic activity). An increase in the activity may indicate that the compound is acting as an agonist, and a decrease may indicate that the compound is an antagonist.

Mimetics are small molecules which are rationally designed to fit a particular target using structural and other information known about proteins. Peptidomimetics are based upon active peptides, but where the number of peptide bonds is reduced.

The polypeptides of the invention or nucleic acids, which encode it, may be used to screen pharmaceuticals. In particular, the angiogenin-4 gene has been found to be a robust and reliable reporter to the presence of gut commensals. Its production therefore can be measured in a screen for compounds that regulate Paneth cell function.

Detection of angiogenin-4 or the other angiogenin-4 mRNA may be carried out using conventional methods. For example the angiogenin-4 gene may be detected using probes or primers. The protein may be detected, for example by immunoassays using antibodies which specifically bind angiogenin-4. Such antibodies are novel and form a further aspect of the invention.

As used herein the term antibody is to be understood to mean a whole antibody or a fragment thereof, for example a F(ab)₂, Fab, FV, VH or VK fragment, a single chain antibody, a multimeric monospecific antibody or fragment thereof, or a bi- or multi-specific antibody or fragment thereof. Each of these types of antibody derivative and their acronyms are well known to the person skilled in the art.

The antibodies for use in this aspect of the invention can be prepared using the angiogenin-4 protein/polypeptides.

Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies which are heterogeneous populations. The term also includes inter alia, humanised and chimeric antibodies.

Monoclonal antibodies to specific antigens may be obtained by methods known to those skilled in the art, such as from hybridoma cells, phage display libraries or other methods. Monoclonal antibodies may be inter alia, human, rabbit or rat derived. For the production of human monoclonal antibodies, hybridoma cells may be prepared by fusing spleen cells from an immunised animal, e.g. a rabbit, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof.

Polyclonal antibodies can be generated by immunisation of an animal (such as a rabbit, rat, goat, horse, sheep etc) see, for example, Example 3.

Rodent antibodies may be humanised using recombinant DNA technology according to techniques known in the art. Alternatively, chimeric antibodies, single chain antibodies, Fab fragments may also be developed against the polypeptides of the invention (Huse et al., Science 256:1275-1281 (1989)), using skills known in the art. Antibodies so produced have a number of uses which will be evident to the molecular biologist or immunologist skilled in the art. Such uses include, but are not limited to, monitoring enzyme expression, development of assays to measure enzyme activity and use as a therapeutic agent. Enzyme linked immunosorbant assays (ELISAs) are well known in the art and would be particularly suitable for detecting the angiogenin-4 protein or polypeptide fragments thereof in a test sample.

In a particular aspect, the invention provides a method for treating inflammatory bowel disease or for preventing and/or treating enteric infections by increasing the levels of a protein of the invention, and particularly angiogenin-4 protein in the intestine of an animal in need thereof. Once again, this can be achieved by administering the protein in a form in which it reaches the intestine, or by other methods as outlined above.

The induction of Ang4 by commensal bacteria is a property that distinguishes it from genes encoding other intestinal microbicidal proteins. The small intestine expresses multiple members of the defensin family, primarily in Paneth cells. Defensin mRNA levels are similar in the intestines of germ-free and conventionally-raised mice (K. Putsep et al. J. Biol. Chem. 275, 40478 (2000). In addition, our previous DNA microarray studies revealed no enhancement in intestinal defensin mRNA expression after B. thetaiotaomicron colonization of germ-free mice (L. V. Hooper et al. Science 291, 881 (2001).

Commensal bacteria contribute to the normal development of adaptive intestinal immune responses (G. L. Talham, et al. Infect. Immun. 67, 1992 (1999). However, little is known about whether, or how, resident microorganisms influence expression of innate mucosal defenses. The results shown here indicate the presence of a heretofore unappreciated mechanism involving regulation of expression/secretion of a small molecular weight, selectively bactericidal Paneth cell protein by components of the microbiota. Microbial regulation of a bactericidal protein during the weaning period represents an elegant way to shape the composition of an evolving mictobiota: obvious feedback elements are present in such a system if both Ang gene expression and bactericidal activity have microbial specificity. Once a climax community is established, the intestine must sustain a stable relationship with its microbiota, avoiding mobilization of immunoinflammatory responses to the complex repertoire of luminal microbial antigens. The advantage of having commensals induce expression of molecules like Ang4 is that access of bacteria to the epithelium is restricted, the chance of activation of cellular immune responses is reduced, especially in ‘sacred spaces’ such as the crypt where tight control of the rate of division of its stem cell and transit amplifying populations is critical, and a ‘hyporesponsive’ state (A. S. Neish et al. Science 289, 1560 (2000)) is perpetuated. Sustaining expression of secreted bactericidal proteins, such as Ang4, by commensal bacteria represents a molecular correlate of the ‘colonization barrier’ that the microbiota provides to prevent encroachment by enteropathogens into the gut ecosystem.

Furthermore, it has been found that angiogenin-4, as well as other members of the angiogenin family, have an antimicrobial activity and therefore, increasing the levels of these proteins in the intestine or even systemically may produce a desirable antibacterial effect.

In a further aspect, the invention provides a method for treating a bacterial or fungal infection in a mammal, which method comprises administering to the mammal an antimicrobial angiogenin, or an antimicrobially active fragment or variant thereof, or an agonist or mimetic thereof, or a compound capable of increasing the amount or activity of an endogous angiogenin protein.

Angiogenins are a class of proteins which are involved in the stimulation of blood vessel development. Examples include human angiogenin of SEQ ID NO 47 as shown in FIG. 1B, as well as the mouse angiogenins. They are a highly conserved series of proteins, which generally have at least 60% sequence identity to SEQ ID NOS 1, 2, 3, 4, or 44, 45, 46 or 47 as shown in FIG. 1.

As used herein, the term “variant” refers to an antimicrobial polypeptides which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will have at least 60% sequence identity, preferably at least 75% sequence identity, and more preferably at least 90% sequence identity to the base sequence.

Sequence identity in this instance can be judged for example using the BLAST P algorithm or the Lipman-Pearson algorithm referred to above.

In a particular embodiment, the angiogenin is a protein of the invention, such as angiogenin-4 as described hereinbefore, but other members of the angiogenin family, and in particular Ang1 of SEQ ID NO 2 or 46 or hAng of SEQ ID NO 47 may also be useful in this context.

In a particularly preferred embodiment, the angiogenin is hang of SEQ ID NO 47 or an antimicrobially active fragment or variant thereof. A particular fragment of SEQ ID NO 47 lacks the N-terminal signal peptide sequence QDNSRY found at the beginning of the sequence. An advantage of using SEQ ID NO 47 of a microbially active fragment thereof is that the protein is of human origin and therefore is unlikely to cause a immune response if administered systemically.

Generally, the angiogenin or derivative thereof will be administered to a human or animal in need of antimicrobial treatment. It may be administered in a conventional manner, for example in the form of a pharmaceutical composition, as would be understood in the art, for example orally or topically. Angiogenins or dervivatives thereof, of human origin may also be administered parenterally.

As used herein, the term “derivatives” include biologically active fragments or variants as defined above, as well as agonists or mimetics or compounds capable of increasing the amount or activity of an endogous angiogenin protein.

They may be used to treat systemic infections, but in particular, these proteins may be used to treat enteric infections of the intestinal tract. Therefore the treatment regime may involve elevating the levels of the proteins in the intestine as outlined previously.

### BRIEF DESCRIPTION OF THE DRAWINGS

FIG. 1A illustrates the sequence alignment of the amino acid sequences of mouse angiogenin family members (SEQ ID NOS 1-4) and FIG. 1B shows the mature protein sequences of some of these (SEQ ID NOS 44-46) compared with the sequence of mature human angiogenin (SEQ ID NO 47).

FIG. 2 shows the nucleotide sequences of mouse angiogenin-4 and angiogenin-3 in alignment (SEQ ID NOS 5 and 6 respectively).

FIG. 3 shows the locations of primers specific for mouse angiogenin family members.

FIG. 4 is a graph illustrating tissue distribution of angiogenin-4 mRNA, together with the results of an agarose gel analysis.

FIG. 5 is a graph illustrating tissue distribution of angiogenin-1 mRNA.

FIG. 6 is a graph illustrating tissue distribution of angiogenin-3 mRNA based on quantitative real-time RT-PCR analysis.

FIG. 7 shows the results of RT-PCR analysis showing the absence of angiogenin-related protein gene expression.

FIG. 8 is a set of graphs showing the results of experiments on the microbial regulation of angiogenin-4 expression in the small intestine.

FIG. 9 is a graph showing the regulation of angiogenin-4 expression during postnatal development.

FIG. 10 is a block graph showing cellular localization of angiogenin-4 expression in small intestine: qRT-PCR analysis of cells isolated from the crypt base.

FIG. 11 shows the results of a bactericidal and fungicidal assay for mouse and human angiogenins.

FIG. 12 shows the results of experiments showing that Ang4 expression is induced by normal intestinal bacteria.

### DETAILED DESCRIPTION OF THE INVENTION

### EXAMPLE 1

Following the observation that a 10d colonization was associated with a 11-fold increase in ileal expression of a mRNA detected by an Affymetrix-designed probe-set designed from the published sequence of angiogenin-3, we designed primers specific for the 3′ and 5′ ends of the mouse angiogenin-3. They were:

| | |
|---|---|
| ORF (forward primer: | |
| 5′-CCTTGGATCCATGGTGATGAGCCCAGGT | (SEQ ID NO 7) |
| TCTTTG | |

which incorporates a BamHI site at the 5′ end; reverse primer:

| | |
|---|---|
| 5′-CCTTTCTAGACTACGGACTGATAAAAGACTCATC | (SEQ ID NO 8) |
| GAAG | |

which incorporates an XbaI site at the 5′ end.

These primers were used together with RT-PCR to amplify a 438 bp sequence from RNA prepared from the ileums of ex-germ-free NMRI mice. These mice had been colonized for 10d with a complete ileal/cecal flora harvested from conventionally-raised animals belonging to the same inbred strain. We subcloned the PCR product into BamHI/XbaI digested pGEX-KG and sequenced it using vector-specific primers.

Surprisingly, the nucleotide sequence of the ORF was only 90% identical to that of mouse angiogenin-3. Since the primer sequences used in the PCR reaction (specific for angiogenin-3) were incorporated into the product, we used 5′- and 3′-RACE to (a) obtain accurate sequence at the 5′ and 3′ ends of the ORF of this new angiogenin, and (b) characterize the 5′- and 3′-untranslated regions of its mRNA. The results revealed only 88.3% nucleotide sequence identity with angiogenin-3 mRNA.

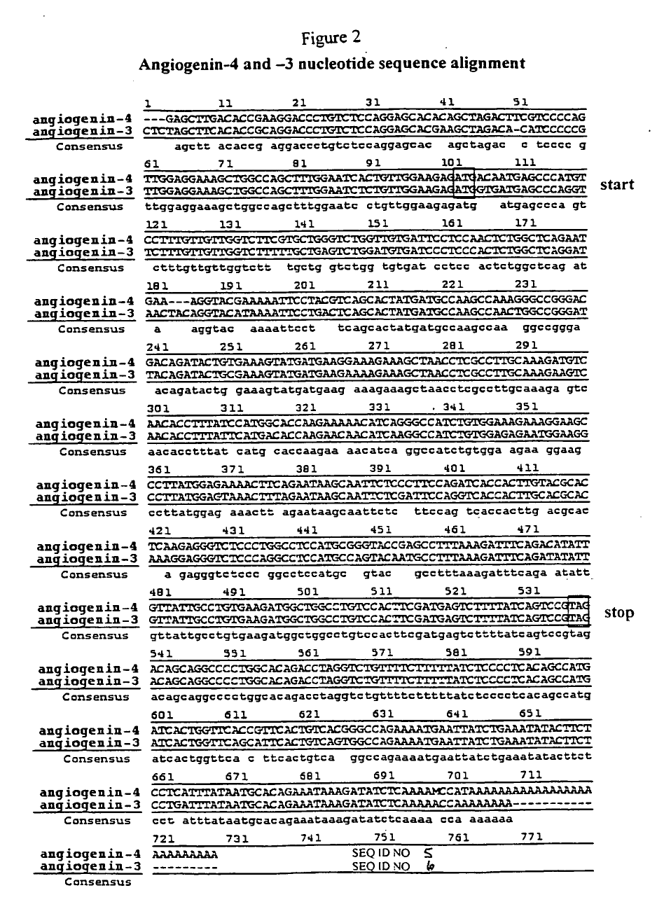

The nucleotide sequence which encodes the angiogenin-4 protein, aligned with the angiogenin-3 sequence is shown hereinafter in FIG. 2 as SEQ ID NO 5 and 6 respectively.

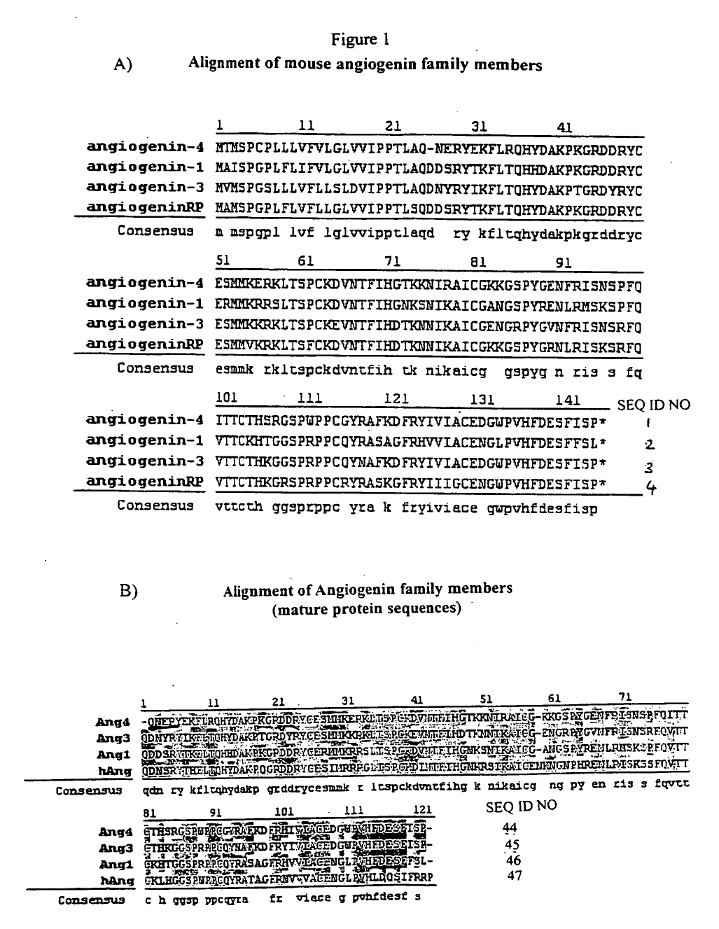

Angiogenin-4 has 74 to 81% amino acid sequence identity to the other 3 members of the mouse angiogenin family (FIG. 1) It was found that the 5′ and 3′-untranslated regions of angiogenin-4 are closely related to the corresponding regions of angiogenin-3 mRNA (FIG. 2).

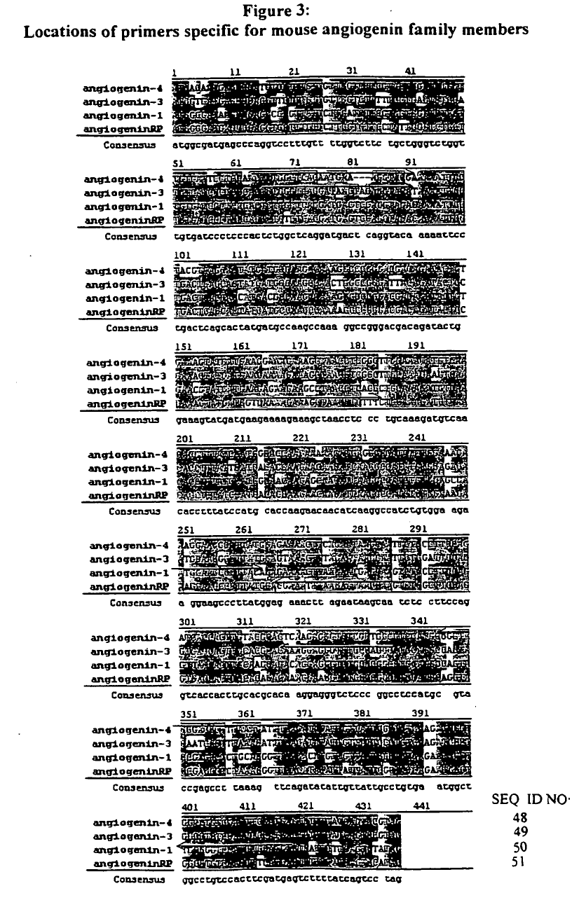

Subsequently a comparative analysis of the tissue distribution of the various mouse angiogenin mRNAs, was conducted. cDNA was synthesized from RNAs isolated from tissues harvested from conventionally raised adult (12-14 week old) male and female NMRI mice (25 tissues/mouse). To quantitate relative levels of expression of each gene, we designed primer sets specific for each of the four mouse angiogenin family members (FIG. 3; Table 1) and used them for SYBR-Green-based real-time quantitative RT-PCR (qRT-PCR) analyses.

TABLE 1

| | | | |
|---|---|---|---|
| | | SEQ ID | |
| Gene | Primer | NO | Sequence |

| | | | |
|---|---|---|---|
| angiogenin-4 | forward | 9 | 5′ CTCTGGCTCAGAATGTAAGGTACGA |
| | reverse | 10 | 5′ GAAATCTTTAAAGGCTCGGTACCC |
| angiogenin-3 | forward | 11 | 5′ CTGGCTCAGGATAACTACAGGTACAT |
| | reverse | 12 | 5′ GCCTGGGAGACCCTCCTTT |
| angiogenin-1 | forward | 13 | 5′ AGCGAATGGAAGCCCTTACA |
| | reverse | 14 | 5′ CTCATCGAAGTGGACCGGCA |
| angiogenin- | forward | 15 | 5′ GGTGAAAAGAAAGCTAACCTCTTTC |
| related | reverse | 16 | 5′ AGACTTGCTTATTCTTAAATTTCG |
| protein | | | |

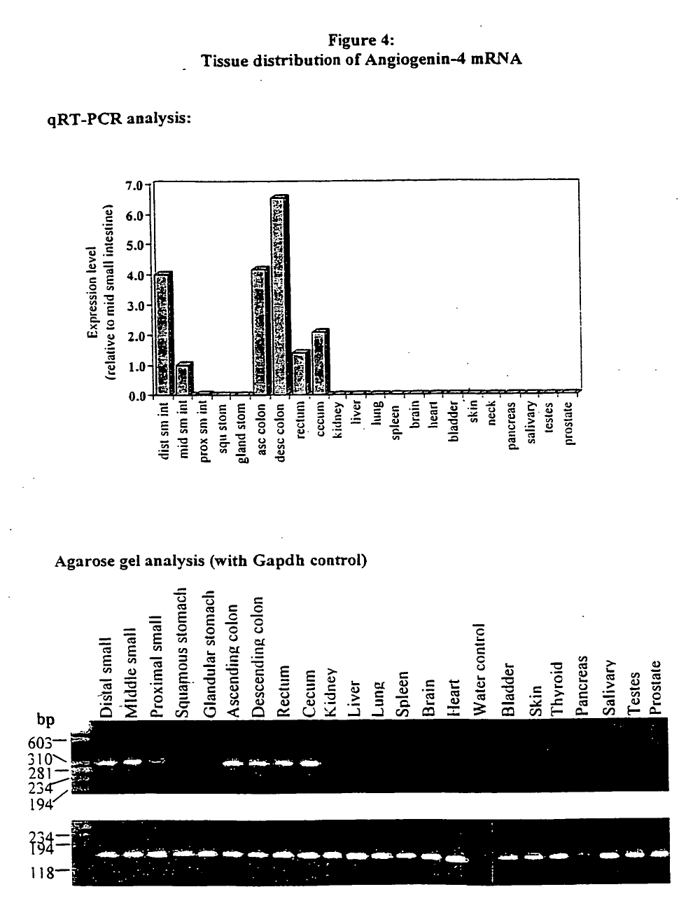

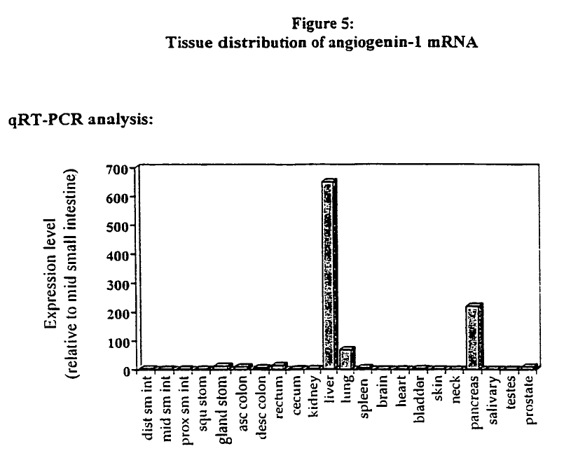

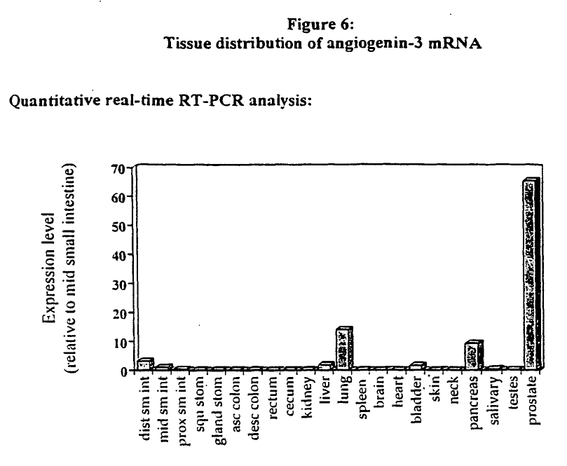

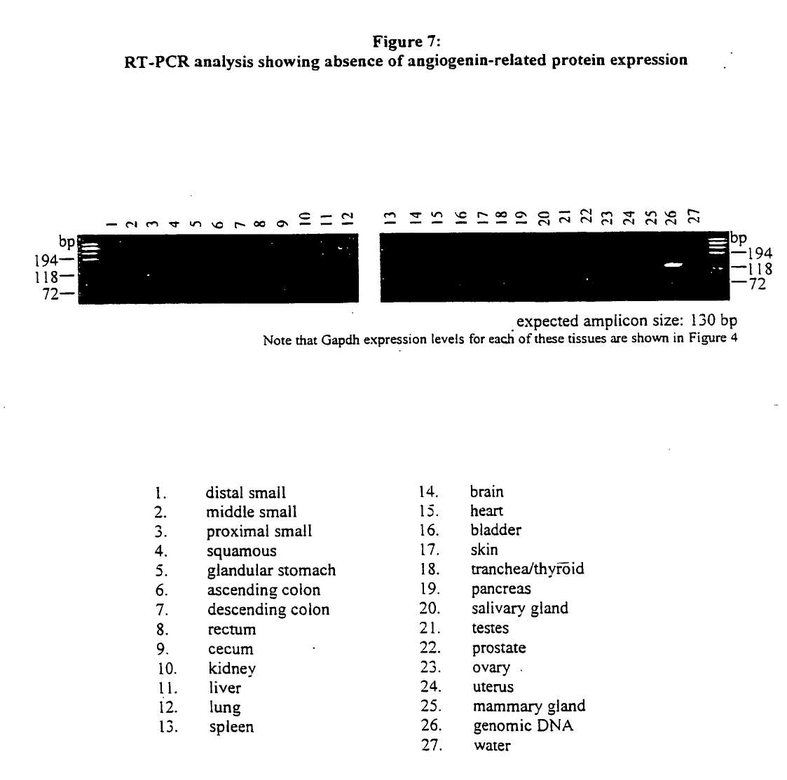

Remarkably, angiogenin-4 mRNA was restricted the intestine where it is expressed from the duodenum to the rectum (FIG. 4). In contrast, angiogenin-1 expression is highest in liver, lung, and pancreas (FIG. 5), while angiogenin-3 is expressed primarily in liver, lung, pancreas, and prostate (FIG. 6). Angiogenin-related protein mRNA was undetectable in all tissues surveyed even after 40 cycles of PCR (FIG. 7).

Thus, the highly restricted, intestine-specific pattern of angiogenin-4 expression makes it unique among mouse angiogenin family members.

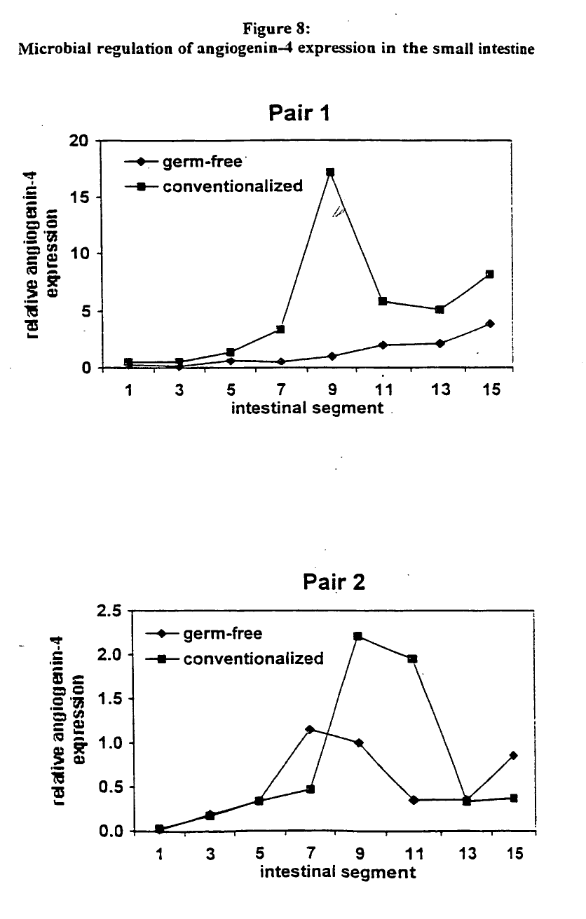

These findings indicated that there was microbial-regulation of angiogenin-4 rather than angiogenin-3 expression in the intestine. To test this hypothesis directly, angiogenin-4-specific primers and qRT-PCR were used to compare angiogenin-4 mRNA levels along the length of the small intestine of germ-free NMRI mice and germ-free mice colonized for 10 d with an ileal/cecal microbiota harvested from conventionally raised NMRI animals. Pair-wise comparisons revealed that expression of angiogenin-4 is highest in the jejunum of colonized mice, and that conventionalization induces up to a 17-fold increase in angiogenin-4 expression in this region (FIG. 8). Mono-association of germ-free NMRI mice with B. thetaiotaomicron for 10d resulted in a comparable induction of angiogenin-4 expression (data not shown).

Regulation of Angiogenin-4 Expression During Postnatal Development is Consistent with its Microbial Regulation

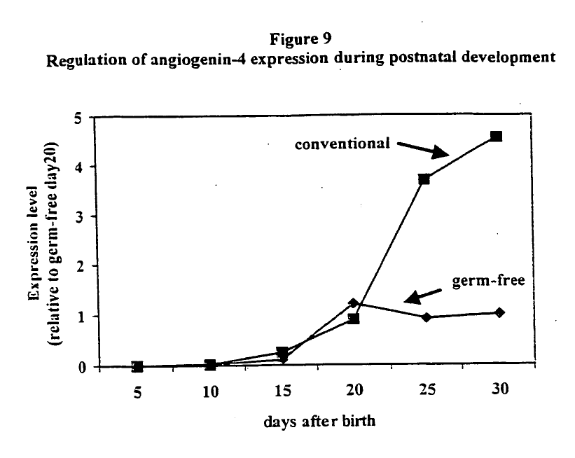

The developmental patterns of angiogenin-4 expression in postnatal day 5 (P5)—P30 germ-free and conventionally raised NMRI mice (n=3 mice per time point per group) was then assessed (FIG. 9). Relative levels of the angiogenin-4 transcript remained relatively low until P20 in both groups of mice. Expression rose slightly (2-3 fold) in germ-free animals after this time point. In contrast, angiogenin-4 expression increased more than 20-fold between P15 and P30 in conventionally-raised animals. These results indicate that angiogenin-4 is induced during the suckling/weaning transition—coincident with a major shift in the gut microbiota. The lack of angiogenin-4 induction in postnatal germ-free mice is also consistent with the conclusion that components of the microbiota play an important role in regulating angiogenin-4 expression in the intestine.

Cellular Localization of Angiogenin-4

The previous laser capture microdissection (LCM)/qRT-PCR study of the cellular origins of angiogenin protein expression (Example 2) used primers that recognize both angiogenin-3 and angiogenin-4, and RNAs that had been isolated from captured crypt epithelium, villus epithelium, or mesenchymal populations from the villus core. The QRT-PCR analysis indicated that the microbially-regulated ‘angiogenin’ was produced in epithelial cells located at the base of crypts of Lieberkuhn (Hooper et al., 2001).

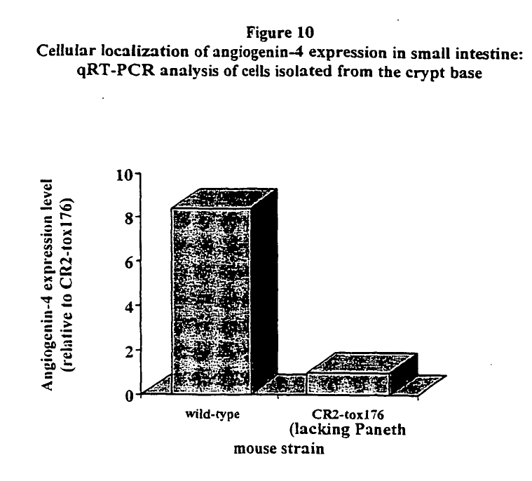

To test the hypothesis that angiogenin-4 expression occurs in Paneth cells, we used LCM to isolate cells located at the base of jejunal crypts from (a) germ-free adult (12 week old) transgenic mice with an attenuated diphtheria toxin-A fragment (tox176)-mediated Paneth cell lineage ablation (CR2-tox176 mice) (Garabedian et al., 1997), and (b) their age and gender-matched germ-free normal littermates. qRT-PCR using angiogenin-4-specific primers revealed that angiogenin-4 mRNA levels are 10-fold higher in RNA purified from crypt base epithelial cells of normal mice compared to CR2-tox176 littermates (FIG. 10).

A follow-up study was conducted using conventionally raised NMRI mice. Three cellular pools were harvested by LCM: Paneth cells alone, epithelial cells from the upper crypt and villus (a Paneth cell-minus fraction), and mesenchyme retrieved from the villus core and the peri-cryptal region. The distribution of angiogenin-4 mRNA closely paralleled the distribution of phospholipase A2—the product of the Mom-1 locus and a well-known Paneth cell-specific gene product (data not shown).

Additional primers used in the qRT-PCR studies are shown in Table 2.

TABLE 2

| | SEQ | | | |
|---|---|---|---|---|
| primer | ID | | | |
| name | NO | sequence | * | purpose |
| Ang3F | 17 | 5′ CCTTGGATCCATGGTGATGAGCCCAGGTTCTTTG | f | Amplification of |
| Ang3R | 18 | 5′ CCTTTCTAGACTACGGACTGATAAAAGACTCATCGAAG | r | Ang4 ORF |
| Ang7 | 19 | 5′ GCCAGGGAGACCCTCTTGA | | 5′ RACE of Ang4 |
| | | | | mRNA |
| Ang10 | 5′ | 5′ CTCTGGCTCAGAATGTAAGGTACGA | | 3′ RACE of Ang4 |
| | | | | mRNA |
| Ang5F | 20 | 5′ AGCGAATGGAAGCCCTTACA | f | qRT-PCR detection |
| Ang7R | 21 | 5′ CTCATCGAAGTGGACCGGCA | r | of mouse Ang1 |
| Ang9 | 22 | 5′ CTGGCTCAGGATAACTACAGGTACAT | f | qRT-PCR detection |
| Ang11 | 23 | 5′ GCCTGGGAGACCCTCCTTT | r | of mouse Ang3 |
| Ang10 | 24 | 5′ CTCTGGCTCAGAATGTAAGGTACGA | f | qRT-PCR detection |
| Ang12 | 25 | 5′ GAAATCTTTAAAGGCTCGGTACCC | r | of mouse Ang4 |
| Arp1F | 26 | 5′ GATACTGCGAAAGTATGATGGT | f | RT-PCR of mouse |
| Arp2R | 27 | 5′ AGACTTGCTTATTCTTAAATTTCG | r | AngRP |
| Arp3F | 28 | 5′ AGAAAGGAAGCCCTTATGGACG | f | RT-PCR of mouse |
| Arp4R | 29 | 5′ CCAGCCATTCTCACAGCCAATAAT | r | AngRP |
| gapdhF | 30 | 5′ TGGCAAAGTGGAGATTGTTGCC | f | qRT-PCR detection |
| gapdhR | 31 | 5′ AAGATGGTGATGGGCTTCCCG | r | of glyceraldehyde |
| | | | | 3- phosphate |
| | | | | dehydrogenase |
| PhosLip | 32 | 5′ CCAAATCACCTGTTCTGCAAAC | f | qRT-PCR detection |
| A2-2F | | | | of secretory |
| PhosLip | 33 | 5′ CATTCAGCGGCGGCTTA | r | phospholipase |
| A2-2R | | | | A₂mRNA |
| Vim-1F | 34 | 5′ TGCTTCTCTGGCACGTCTTG | f | qRT-PCR detection |
| Vim-1R | 35 | 5′ GGACATGCTGTTCCTGAATCTG | r | of vimentin mRNA |
| Keratn4 | 36 | 5′ GCTGAAGTTCGTGCCCAGTAC | f | qRT-PCR detection |
| F | | | | of keratin-8 mRNA |
| Keratn4 | 37 | 5′ CTTTGTGCGGCGCAGAT | r | |
| R | | | | |

### EXAMPLE 2

### Expression and Purification of Angiogenins Including Angiogenin-4 for Functional Assays

pET3a-based expression vectors have been constructed for expression of angiogenin-4 and angiogenin-1 (positive control) in E. coli. Specifically, ORFs encoding mouse Ang1, mouse Ang4, and human Ang ((add ref) were amplified by RT-PCR using cDNAs from adult mouse liver, adult mouse mid-small intestine (jejunum), and human small intestine, respectively, plus gene specific primers (see Table 3).

TABLE 2

| | SEQ | | | |
|---|---|---|---|---|
| primer | ID | | | |
| name | NO | sequence | * | purpose |
| Ang25 | 38 | 5′ GGGAATTCCATATGCAGAATGAAAGGTACGAAAAATTCCTAC | f | construction of |
| Ang23 | 39 | 5′ CCTTGGATCCCTACGGACTGATAAAAGACTCATCG | r | pET3a-[met⁻¹]Ang4 |
| | | | | (expression |
| | | | | construct) |
| Ang27 | 40 | 5′ GGGAATTCCATATGCAGGATGACTCCAGGTACACAAAATT | f | construction of |
| Ang24 | 41 | 5′ CCTTGGATCCCTATAGACTGAAAAATGACTCATCGAAG | r | pET3a-[met⁻¹]Ang1 |
| | | | | (expression |
| | | | | construct) |
| Ang30 | 42 | 5′ GGGAATTCCATATGCAGGATAACTCCAGGTACACACACTT | f | construction of |
| Ang29 | 43 | 5′ CCTTGGATCCTTACGGACGACGGAAAATTGACTGA | r | pET3a-[met⁻¹]hAng |
| | | | | (expression |
| | | | | construct) |

The resulting amplicons contained a Met codon in place of the signal sequence, and incorporated 5′ Nde I and 3′ Bam HI sites. PCR products were digested with Nde I and Bam HI, cloned into Nde I/Bam HI-digested pET3a (Invitrogen), and sequenced.

The recombinant plasmids were then introduced into E. coli BL21-CodonPlus (DE3)—RIL cells (Stratagene). Protein expression was induced in mid-log phase cultures with 0.5 mm isopropylthiogalactoside using a protocol developed by the manufacturer (Stratagene). Cells were subsequently harvested by centrifugation (6500×g)₁ dispersed in 0.1 volume IB Buffer (20 mM Tris-HCl pH 7.5, 10 mM EDTA, 1% Triton X-100) containing 100 μg/ml lysozyme, and disrupted by sonication.

Preliminary experiments showed that following IPTG induction, each angiogenin was localized in inclusion bodies. This feature is a common property of members of the angiogenin family.

Inclusion bodies were collected by centrifugation at 10,000×g, washed twice in IB Buffer, and solubilized in 7 M guanidine-HCl, 0.15 M reduced glutathione, 0.1 M Tris-HCl (pH 8), and 2 mM EDTA. Angiogenins were refolded and purified by cation-exchange chromatography (S2). The purity of each protein preparation was assessed by SDS-PAGE and by N-terminal sequencing. RNase activity (S3) was measured to determine that proteins had refolded properly. Angiogenins were dialyzed against 10 mM sodium phosphate (pH 7.2).

Proper folding can be assessed by solubility and by assay for RNase activity (a shared feature of angiogenins).

### EXAMPLE 3

Generation of Polyclonal Antibodies

Purified angiogenin-4 was used to generate polyclonal antibodies in rabbits. Although the antibodies may also recognize other angiogenins, since angiogenin-4 is the only angiogenin produced in the intestinal epithelium, these antibodies will be very useful for examining its trafficking in Paneth cells (see below).

### EXAMPLE 4

Assays for Angiogenic Activity

The standard biological assay for angiogenic activity uses the chick chorioallantoic membrane. Various amounts of purified angiogenin-1 (positive control) or angiogenin-4 can be applied to the membrane and new blood vessel formation scored in single-blinded fashion.

It is possible also to generate 3-D reconstructions of the peri-cryptal and villus vascular network in several mouse models using a novel florochrome-labeled angiographic assay (Hashimoto, H., et al. (1998) Microvascular Res. 55:179, Stappenbeck T. S., Hooper L. V. & Gordon J. I (2002) Proc. Natl. Acad. Sci USA, 99, 15451-15455). This assay employs confocal laser scanning microscopy of 120 μm thick sections of intestine harvested from mice that have been subjected to ventricular perfusion with a solution of either fluroescein-conjugated high molecular weight dextran or rhodamine B-labeled gelatin. Two sets of comparisons may be made: (a) adult germ-free FVB/N CR2-tox176 transgenic versus normal littermates (i.e. animals without and with Paneth cells in jejunal crypts respectively); (b) adult germ-free NMRI mice versus age and gender-matched ex-germ-free animals colonized for 10 and 35 d with an unfractionated ileal/cecal microbiota.

### EXAMPLE 5

Assays for Bacteriostatic or Bactericidal Activity

Purified angiogenin-4 can be assayed for bactericidal activity using conventional methods. For instance, it can be incubated with members of the normal intestinal flora (e.g., E. coli, B. thetaiotaomicron, Clostridium spp., Lactobacillus spp.), various intestinal pathogens (e.g., S. typhimurium, Shigella flexneri, Campylobacter jejuni). A RNase with known bactericidal activity is suitably used as a positive control (human eosinophil cationic protein, Lehrer, R. I., et al. (1989) J. Immunol. 142, 4428-4434).

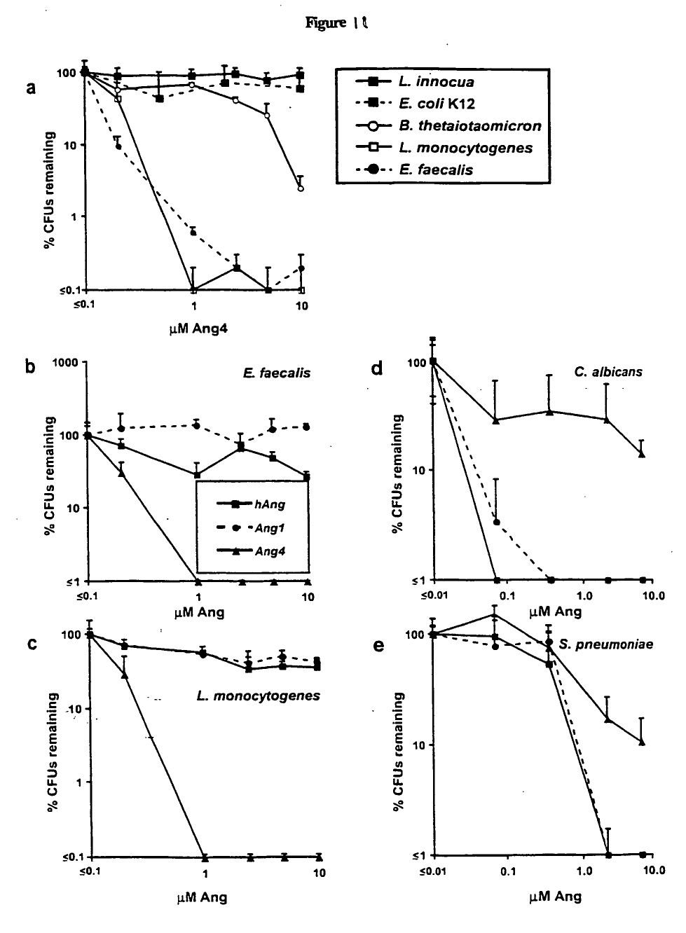

Purified angiogenin-4, obtained as described in Example 2, was assayed for bactericidal activity against several Gram-positive and Gram-negative intestinal pathogens and commensals. The percentage of colony forming units (CFUs) remaining after exposure to increasing concentrations of purified Ang4 was determined and is shown in FIG. 11A. Bacteria were grown to mid-log phase and resuspended in 10 mM sodium phosphate buffer (pH 7.2). Initial bacterial concentrations ranged from 10⁵ to 10⁶ CFU/ml. After incubation for 2 hours at 37° C., viable bacteria were quantitated by dilution plating. Assays were done in triplicate. Mean values±SD were plotted and are shown in FIG. (11A) Comparison of bactericidal activity of purified hang, Ang1, and Ang4 against (B) Enterococcus faecalis (ATCC 29212), (C) Listeria monocytogenes (strain EGD-e; P. Glaser et al. Science 294, 849 (2001), (D) Candida albicans, and (E) Streptococcus pneumoniae are also shown.

The number of colony forming units of log-phase Enterococcus faecalis or Listeria monocytogenes (both Gram-positive pathogens) declined by >99% after a 2 h exposure to 1 μM Ang4 (FIG. 11A). The intestinal commensal B. thetaiotaomicron was less sensitive: the number of viable organisms was reduced 30% (mean±1 SD) with 1 μM Ang4. Another Gram-negative commensal, Escherichia coli K12, was resistant to 10 μM Ang4 (FIG. 11A). Despite its close genetic relationship to L. monocytogenes (Glaser et al. supra.), Listeria innocua was resistant to Ang4 at concentrations up to 10 μM (FIG. 11A), thereby emphasizing that species-specific features mediate susceptibility or resistance to this host-derived anti-microbial protein.

Approximately equivalent amounts of immunoreactive lysozyme and Ang4 were detected in Paneth cell secretory granules (not shown). Assuming that Ang4 is secreted into the crypt lumen in vivo at levels similar to those of other Paneth cell granule proteins (T. Ayabe et al. Nat. Immunol. 1, 113 (2000), D. Ghosh et al. Nat. Immunol. 3, 583 (2002)), its estimated concentration in the crypt (>1 mM) would be 1000 times greater than that required to kill E. faecalis or L. monocytogenes (FIG. 11A,E).

Ang1 and hAng were investigated to see whether they could function as mediators of systemic innate responses to infection, since they appear in the bloodstream during the acute phase response to infection (K. A. Olson, S. J. Verselis, J. W. Fett. Biochem. Biophys. Res. Commun. 242, 480 (1998)). In contrast to the potent bactericidal activity exhibited by Ang4 towards E. faecalis and L. monocytogenes, both bacterial species were resistant to Ang1 and hang (FIG. 11B,C). However two organisms that cause systemic infection in humans were sensitive to Ang1 and hAng. Candida albicans, an opportunistic fungal pathogen, exhibited a 97% and a >99% decline in CFU 2 hours after exposure to 70 nM of Ang1 and hAng—a concentration that is comparable to their levels in serum.

Likewise, 2 μM Ang1 or hAng produced a >99% decline in the viability of Steptococcus pneumoniae, the Gram-positive pathogen that commonly causes pneumonia and sepsis. Both of these pathogens were considerably less sensitive to Ang4 (FIG. 11D, E) Thus, mouse Ang1 and hAng, which share 77% amino acid sequence identity, appear to represent previously unappreciated components of systemic innate host defense to infection.

### EXAMPLE 6

LCM Studies of the Mouse Colon and Human Gut

LCM can be used to identify the cellular origin of angiogenin-4 in the adult mouse colon (which lacks Paneth cells). Cells are dissected from three domains: the lower third of the crypt, the remaining upper two thirds of the crypt, and from the surface epithelial cuff (colonic homolog of the villus). Adult germ-free and conventionally raised NMRI mice were then used in the experiment, although other strains would have been useful. Expression can be assayed by qRT-PCR using primers specific for angiogenins 1, 3, and 4. As angiogenin-4 is the only angiogenin detected in the crypt epithelium, then the antibodies prepared as described above in Example 3 can be used for further analysis of the cellular pattern of expression.

LCM/qRT-PCR can also be used to compare and contrast angiogenin-4 expression in Apc^{Min}/+ mice: RNA from adenomas and adjacent normal appearing epithelium can be assayed.

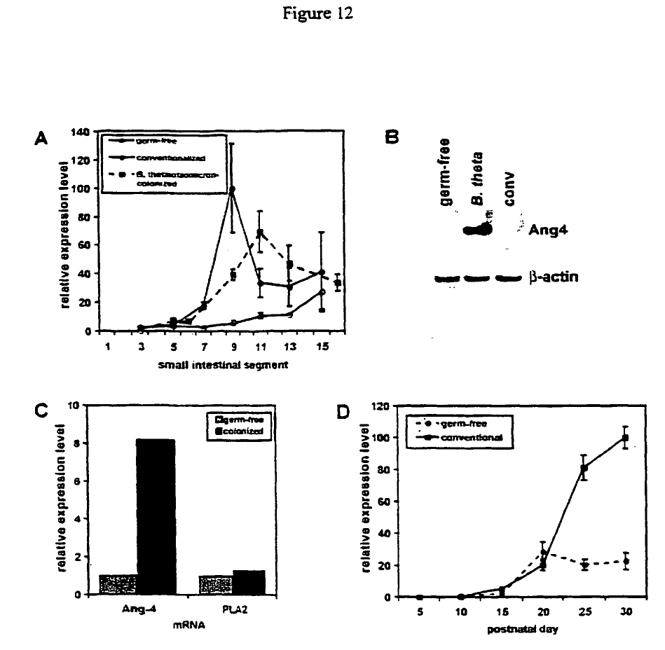

Specifically, several observations indicate that Ang4 expression regulated in vivo by components of the intestinal microbiota. A 10 d colonization of adult germ-free NMRI mice with an unfractionated microbiota harvested from the distal small intestine and cecums of age- and gender-matched conventionally raised animals, elicits an increase in Ang4 mRNA concentrations throughout the small bowel. Small intestines from germ-free or colonized (L. V. Hooper et al. Science 291, 881 (2001)) mice were divided into 16 equal segments and Ang4 mRNA levels determined in each segment using qRT-PCR (triplicate assays; mean values±SD plotted This response is recapitulated by colonization with B. thetaiotaomicron alone, indicating that Ang4 expression is enhanced by at least one normal member of the microbiota (FIG. 12A)). Results as shown in FIG. 12A, are representative of three independent experiments.

qRT-PCR study showing colonization-associated enhancement of Ang4 but not sPLA₂ mRNA expression in microdissected Paneth cells. Bacterial induction of Ang4 mRNA in laser capture microdissected Paneth cells harvested from B. thetaiotaomicron-monoassociated and conventionalized mice was confirmed (e.g., FIG. 12C). In contrast, sPLA₂ mRNA levels were unaffected (FIG. 12C), indicating that the microbe-dependent increase in Ang4 expression is not part of a generalized induction of genes encoding Paneth cell secretory granule proteins.

Immunoblot analysis of 25 μg of total cellular protein isolated from small intestinal segment 9 was carried out. The blot was probed with anti-Ang, then stripped and re-probed with anti-β-actin and showed increased Ang4 protein levels in B. thetaiotaomicron-colonized mice. This increase parallels the rise in its mRNA (FIG. 12B). The dramatic increase in Ang4 protein was not observed in the intestines of mice colonized for 10 d with a complete distal intestinal microbiota (FIG. 12B; note that the density of colonization, as measured by CFU, was equivalent in the two groups of animals). This difference may be due to translational or post-translational regulation of Ang4 expression, or enhanced de-granulation of Paneth cells in conventionalized as compared to B. thetaiotaomicron-monoassociated intestines.

qRT-PCR assays of Ang4 expression during postnatal development provided additional evidence supporting its microbial regulation. Assays were performed on pooled mid-small intestinal RNAs (n=3 mice/time point). All qRT-PCR determinations were done in triplicate.

Ang4 mRNA levels rose markedly in the small intestines of conventionally-raised but not germ-free NMRI mice during the weaning period (postnatal days 17-28; FIG. 12D). Weaning is associated with a dramatic change in the composition of the gut microbiota, as the abundance of Gram-positive and -negative facultative anaerobes declines and Gram-negative obligate anaerobes become predominant (D. C. Savage. Annu. Rev. Microbiol. 31, 107 (1977)).

1. An isolated polypeptide comprising SEQ ID NO 1 as shown in FIG. 1 hereinafter, or an allelic variant thereof or a polypeptide which has at least 85% amino acid sequence identity with SEQ ID NO 1, or a biologically active fragment of any of these.

2. An isolated polypeptide according to claim 1 which has at least 95% amino acid sequence identity with SEQ ID NO 1 or a or a biologically active fragment of any of these.

3. An isolated polypeptide according to claim 1 which comprises SEQ ID NO 1 or a biologically active fragment thereof.

4. An isolated polypeptide according to claim 3 which comprises a fragment of SEQ ID NO 1 which is SEQ ID NO 44.

5. An isolated polypeptide according to claim 3 which comprises a fragment of SEQ ID NO 1 which is SEQ ID NO 44 but which lacks the first five amino acids.

6. An isolated nucleic acid which encodes a polypeptide according to claim 1.

7. A nucleic acid according to claim 6 which comprises at least part of SEQ ID NO 5 as shown in FIG. 2.

8. A plasmid comprising a nucleic acid according to claim 6 or claim 7.

9. A vector comprising a nucleic acid according to claim 6 or claim 7.

10. A cell which comprises a plasmid according to claim 8 or a vector containing said nucleic acid.

11. A method of modifying the angiogenic activity within the intestine of a human or animal in need thereof, which method comprises affecting the activity of a polypeptide according to claim 1 within the intestine.

12. A method of modifying the angiogenic activity within the intestine thereof a human or animal in need of comprising administering a polypeptide according to claim 1 or a mimetic thereof, to the intestine.

13. A method according to claim 12 wherein the polypeptide administered is SEQ ID NO 44 or a polypeptide of SEQ ID NO 44 which lacks the first 5 amino acids of SEQ ID NO 44.

14. A method according to claim 11 wherein the animal produces angiogenin-4 and the activity is enhanced by administration of a polypeptide or the invention, or an agonist or mimetic thereof into the intestine.

15. A method according to claim 11 which comprises administration to the animal intestine of a microorganism which regulates the expression of angiogenin-4 or a chemical entity identified as being produced by components of the microbiota.

16. A method according to claim 15 wherein the microorganism is B. thetaiotaomicron.

17. A method according to claim 11 where the activity of angiogenin-4 is reduced by administering an antagonist of protein activity, or a compound or antibody, which blocks the activity of the protein by binding to it and inactivating it.

18. A method according to claim 11 where the activity of angiogenin-4 is reduced by administration of an antisense DNA or RNA construct which hydridises to angiogenin-4 DNA or RNA respectively, or a ribozyme molecule which specifically cleaves Ang4 mRNA, or an RNA interference (RNAi) oligonucleotide which is specific for an Ang4 sequence.

19. A method for treating inflammatory bowel disease or preventing or treating enteric infections by increasing the levels of a polypeptide according to claim 1 in the intestine of an animal in need thereof.

20. A pharmaceutical composition comprising a polypeptide according to claim 1 or an agonist, antagonist or mimetic thereof, in combination with a pharmaceutically acceptable carrier.

21. A composition according to claim 20 wherein the formulation is provided with an enteric coating.

22. A method for screening for compounds which regulate Paneth cell function, said method comprising contacting said compounds with mouse Paneth cells, and detecting angiogenin-4 or the angiogenin-4 gene.

23. An antibody which specifically binds to a polypeptide according to claim 1.

24. A method for treating a bacterial or fungal infection in a mammal, which method comprises administering to the mammal a polypeptide comprising an antimicrobial angiogenin, or an antimicrobially active fragment or variant thereof, or an agonist or mimetic thereof, or a compound capable of increasing the amount or activity of an endogous angiogenin protein.

25. A method according to claim 24 wherein the antimicrobial angiogenin is of SEQ ID NOS 1, 2, 3, 4, or 44, 45, 46 or 47 as shown in FIG. 1, or an antimicrobial variant having at least 60% sequence identity to any of these.

26. A method for treating a bacterial or fungal infection in a mammal, which method comprises administering to the mammal a polypeptide comprising a polypeptide according to claim 1.

27. A method according to claim 24 wherein the polypeptide is a polypeptide of SEQ ID NO 2 or 46.

28. A method according to claim 24 wherein the polypeptide is a polypeptide of SEQ ID NO 47 or an antimicrobially active fragment or variant thereof.

29. A method according to claim 28 wherein the polypeptide is a fragment of SEQ ID NO 47 that lacks the N-terminal signal peptide sequence QDNSRY (SEQ ID NO: 57) found at positions 1-6 of the sequence.

30. A method according to claim 29 wherein the angiogenin is administered parenterally to a human.

31. A method according to claim 28 for the treatment of fungal infections.

32. A method according to claim 24 wherein the polypeptide is administered orally.

33. A method according to claim 32 for the treatment of bacterial or fungal infections of the intestinal tract.


SEQUENCE LISTING (57 sequences)

SEQ ID NO: 1 - protein, 144 aa, Mus sp.
MTMSPCPLLLVFVLGLVVIPPTLAQNERYEKFLRQHYDAKPKGRDDRYCESMMKERKLTSPCKDVNTFIHGTKKNIRAICGKKGSPYGENFRISNSPFQITTCTHSRGSPWPPCGYRAFKDFRYIVIACEDGWPVHFDESFISP

SEQ ID NO: 2 - protein, 145 aa, Mus sp.
MAISPGPLFLIFVLGLVVIPPTLAQDDSRYTKFLTQHHDAKPKGRDDRYCERMMKRRSLTSPCKDVNTFIHGNKSNIKAICGANGSPYRENLRMSKSPFQVTTCKHTGGSPRPPCQYRASAGFRHVVIACENGLPVHFDESFFSL

SEQ ID NO: 3 - protein, 145 aa, Mus sp.
MVMSPGSLLLVFLLSLDVIPPTLAQDNYRYIKFLTQHYDAKPTGRDYRYCESMMKKRKLTSPCKEVNTFIHDTKNNIKAICGENGRPYGVNFRISNSRFQVTTCTHKGGSPRPPCQYNAFKDFRYIVIACEDGWPVHFDESFISP

SEQ ID NO: 4 - protein, 145 aa, Mus sp.
MAMSPGPLFLVFLLGLVVIPPTLSQDDSRYTKFLTQHYDAKPKGRDDRYCESMMVKRKLTSFCKDVNTFIHDTKNNIKAICGKKGSPYGRNLRISKSRFQVTTCTHKGRSPRPPCRYRASKGFRYIIIGCENGWPVHFDESFISP

SEQ ID NO: 5 - DNA, 722 bp, Mus sp.
gagcttgacaccgaaggaccctgtctccaggagcacacagctagactcgtccccagttggaggaaagctggccagctttggaatcactgttggaagagatgacaatgagcccatgtcctttgttgttggtcttcgtgctgggtctggttgtgattcctccaactctggctcagaatgaaaggtacgaaaaattcctacgtcagcactatgatgccaagccaaagggccgggacgacagatactgtgaaagtatgatgaaggaaagaaagctaacctcgccttgcaaagatgtcaacacctttatccatggcaccaagaaaaacatcagggccatctgtggaaagaaaggaagcccttatggagaaaacttcagaataagcaattctcccttccagatcaccacttgtacgcactcaagagggtctccctggcctccatgcgggtaccgagcctttaaagatttcagatatattgttattgcctgtgaagatggctggcctgtccacttcgatgagtcttttatcagtccgtagacagcaggcccctggcacagacctaggtctgttttctttttatctcccctcacagccatgatcactggttcaccgttcactgtcacgggccagaaaatgaattatctgaaatatacttctcctcatttataatgcacagaaataaagatatctcaaaamccataaaaaaaaaaaaaaaaaaaaaaaaaa

SEQ ID NO: 6 - DNA, 708 bp, Mus sp.
ctctagcttcacaccgcaggaccctgtctccaggagcacgaagctagacacatcccccgttggaggaaagctggccagctttggaatctctgttggaagagatggtgatgagcccaggttctttgttgttggtctttttgctgagtctggatgtgatccctcccactctggctcaggataactacaggtacataaaattcctgactcagcactatgatgccaagccaactggccgggattacagatactgcgaaagtatgatgaagaaaagaaagctaacctcgccttgcaaagaagtcaacacctttattcatgacaccaagaacaacatcaaggccatctgtggagagaatggaaggccttatggagtaaactttagaataagcaattctcgattccaggtcaccacttgcacgcacaaaggagggtctcccaggcctccatgccagtacaatgcctttaaagatttcagatatattgttattgcctgtgaagatggctggcctgtccacttcgatgagtcttttatcagtccgtagacagcaggcccctggcacagacctaggtctgttttctttttatctcccctcacagccatgatcactggttcagcattcactgtcagtggccagaaaatgaattatctgaaatatacttctcctgatttataatgcacagaaataaagatatctcaaaaaccaaaaaaaa

SEQ ID NO: 7 - DNA, 34 bp, Artificial Sequence
  Description of Artificial Sequence Primer
ccttggatccatggtgatgagcccaggttctttg

SEQ ID NO: 8 - DNA, 38 bp, Artificial Sequence
  Description of Artificial Sequence Primer
cctttctagactacggactgataaaagactcatcgaag

SEQ ID NO: 9 - DNA, 25 bp, Artificial Sequence
  Description of Artificial Sequence Primer
ctctggctcagaatgtaaggtacga

SEQ ID NO: 10 - DNA, 24 bp, Artificial Sequence
  Description of Artificial Sequence Primer
gaaatctttaaaggctcggtaccc

SEQ ID NO: 11 - DNA, 26 bp, Artificial Sequence
  Description of Artificial Sequence Primer
ctggctcaggataactacaggtacat

SEQ ID NO: 12 - DNA, 19 bp, Artificial Sequence
  Description of Artificial Sequence Primer
gcctgggagaccctccttt

SEQ ID NO: 13 - DNA, 20 bp, Artificial Sequence
  Description of Artificial Sequence Primer
agcgaatggaagcccttaca

SEQ ID NO: 14 - DNA, 20 bp, Artificial Sequence
  Description of Artificial Sequence Primer
ctcatcgaagtggaccggca

SEQ ID NO: 15 - DNA, 25 bp, Artificial Sequence
  Description of Artificial Sequence Primer
ggtgaaaagaaagctaacctctttc

SEQ ID NO: 16 - DNA, 24 bp, Artificial Sequence
  Description of Artificial Sequence Primer
agacttgcttattcttaaatttcg

SEQ ID NO: 17 - DNA, 34 bp, Artificial Sequence; identical to SEQ ID NO: 7
  Description of Artificial Sequence Primer

SEQ ID NO: 18 - DNA, 38 bp, Artificial Sequence; identical to SEQ ID NO: 8
  Description of Artificial Sequence Primer

SEQ ID NO: 19 - DNA, 19 bp, Artificial Sequence
  Description of Artificial Sequence Primer
gccagggagaccctcttga

SEQ ID NO: 20 - DNA, 20 bp, Artificial Sequence
  Description of Artificial Sequence Primer
agcgaatggaagcccttaca

SEQ ID NO: 21 - DNA, 20 bp, Artificial Sequence
  Description of Artificial Sequence Primer
ctcatcgaagtggaccggca

SEQ ID NO: 22 - DNA, 26 bp, Artificial Sequence
  Description of Artificial Sequence Primer
ctggctcaggataactacaggtacat

SEQ ID NO: 23 - DNA, 19 bp, Artificial Sequence
  Description of Artificial Sequence Primer
gcctgggagaccctccttt

SEQ ID NO: 24 - DNA, 25 bp, Artificial Sequence
  Description of Artificial Sequence Primer
ctctggctcagaatgtaaggtacga

SEQ ID NO: 25 - DNA, 24 bp, Artificial Sequence
  Description of Artificial Sequence Primer
gaaatctttaaaggctcggtaccc

SEQ ID NO: 26 - DNA, 22 bp, Artificial Sequence
  Description of Artificial Sequence Primer
gatactgcgaaagtatgatggt

SEQ ID NO: 27 - DNA, 24 bp, Artificial Sequence
  Description of Artificial Sequence Primer
agacttgcttattcttaaatttcg

SEQ ID NO: 28 - DNA, 22 bp, Artificial Sequence
  Description of Artificial Sequence Primer
agaaaggaagcccttatggacg

SEQ ID NO: 29 - DNA, 24 bp, Artificial Sequence
  Description of Artificial Sequence Primer
ccagccattctcacagccaataat

SEQ ID NO: 30 - DNA, 22 bp, Artificial Sequence
  Description of Artificial Sequence Primer
tggcaaagtggagattgttgcc

SEQ ID NO: 31 - DNA, 21 bp, Artificial Sequence
  Description of Artificial Sequence Primer
aagatggtgatgggcttcccg

SEQ ID NO: 32 - DNA, 22 bp, Artificial Sequence
  Description of Artificial Sequence Primer
ccaaatcacctgttctgcaaac

SEQ ID NO: 33 - DNA, 17 bp, Artificial Sequence
  Description of Artificial Sequence Primer
cattcagcggcggctta

SEQ ID NO: 34 - DNA, 20 bp, Artificial Sequence
  Description of Artificial Sequence Primer
tgcttctctggcacgtcttg

SEQ ID NO: 35 - DNA, 22 bp, Artificial Sequence
  Description of Artificial Sequence Primer
ggacatgctgttcctgaatctg

SEQ ID NO: 36 - DNA, 21 bp, Artificial Sequence
  Description of Artificial Sequence Primer
gctgaagttcgtgcccagtac

SEQ ID NO: 37 - DNA, 17 bp, Artificial Sequence
  Description of Artificial Sequence Primer
ctttgtgcggcgcagat

SEQ ID NO: 38 - DNA, 42 bp, Artificial Sequence
  Description of Artificial Sequence Primer
gggaattccatatgcagaatgaaaggtacgaaaaattcctac

SEQ ID NO: 39 - DNA, 35 bp, Artificial Sequence
  Description of Artificial Sequence Primer
ccttggatccctacggactgataaaagactcatcg

SEQ ID NO: 40 - DNA, 40 bp, Artificial Sequence
  Description of Artificial Sequence Primer
gggaattccatatgcaggatgactccaggtacacaaaatt

SEQ ID NO: 41 - DNA, 38 bp, Artificial Sequence
  Description of Artificial Sequence Primer
ccttggatccctatagactgaaaaatgactcatcgaag

SEQ ID NO: 42 - DNA, 40 bp, Artificial Sequence
  Description of Artificial Sequence Primer
gggaattccatatgcaggataactccaggtacacacactt

SEQ ID NO: 43 - DNA, 35 bp, Artificial Sequence
  Description of Artificial Sequence Primer
ccttggatccttacggacgacggaaaattgactga

SEQ ID NO: 44 - protein, 120 aa, Mus sp.
QNERYEKFLRQHYDAKPKGRDDRYCESMMKERKLTSPCKDVNTFIHGTKKNIRAICGKKGSPYGENFRISNSPFQITTCTHSRGSPWPPCGYRAFKDFRYIVIACEDGWPVHFDESFISP

SEQ ID NO: 45 - protein, 121 aa, Mus sp.
QDNYRYIKFLTQHYDAKPTGRDYRYCESMMKKRKLTSPCKEVNTFIHDTKNNIKAICGENGRPYGVNFRISNSRFQVTTCTHKGGSPRPPCQYNAFKDFRYIVIACEDGWPVHFDESFISP

SEQ ID NO: 46 - protein, 121 aa, Mus sp.
QDDSRYTKFLTQHHDAKPKGRDDRYCERMMKRRSLTSPCKDVNTFIHGNKSNIKAICGANGSPYRENLRMSKSPFQVTTCKHTGGSPRPPCQYRASAGFRHVVIACENGLPVHFDESFFSL

SEQ ID NO: 47 - protein, 123 aa, Mus sp. [sequence omitted]

SEQ ID NO: 48 - DNA, 435 bp, Mus sp.
atgacaatgagcccatgtcctttgttgttggtcttcgtgctgggtctggttgtgattcctccaactctggctcagaatgaaaggtacgaaaaattcctacgtcagcactatgatgccaagccaaagggccgggacgacagatactgtgaaagtatgatgaaggaaagaaagctaacctcgccttgcaaagatgtcaacacctttatccatggcaccaagaaaaacatcagggccatctgtggaaagaaaggaagcccttatggagaaaacttcagaataagcaattctcccttccagatcaccacttgtacgcactcaagagggtctccctggcctccatgcgggtaccgagcctttaaagatttcagatatattgttattgcctgtgaagatggctggcctgtccacttcgatgagtcttttatcagtccgtag

SEQ ID NO: 49 - DNA, 438 bp, Mus sp.
atggtgatgagcccaggttctttgttgttggtctttttgctgagtctggatgtgatccctcccactctggctcaggataactacaggtacataaaattcctgactcagcactatgatgccaagccaactggccgggattacagatactgcgaaagtatgatgaagaaaagaaagctaacctcgccttgcaaagaagtcaacacctttattcatgacaccaagaacaacatcaaggccatctgtggagagaatggaaggccttatggagtaaactttagaataagcaattctcgattccaggtcaccacttgcacgcacaaaggagggtctcccaggcctccatgccagtacaatgcctttaaagatttcagatatattgttattgcctgtgaagatggctggcctgtccacttcgatgagtcttttatcagtccgtag

SEQ ID NO: 50 - DNA, 438 bp, Mus sp.
atggcgataagcccaggcccgttgttcttgatcttcgtgctgggtctggttgtgatccctcccactctggctcaggatgactccaggtacacaaaattcctgactcagcaccatgacgccaagccaaagggccgggacgacagatactgtgaacgtatgatgaagagaagaagcctaacctcaccctgcaaagatgtcaacacctttatccatggcaacaagagcaacatcaaggccatctgtggagcgaatggaagcccttacagagaaaacttaagaatgagcaagtctcccttccaggtcaccacttgcaagcacacaggagggtctccccggcctccatgccagtaccgagcctctgcagggttcagacatgttgttattgcctgtgagaatggcttgccggtccacttcgatgagtcatttttcagtctatag

SEQ ID NO: 51 - DNA, 438 bp, Mus sp.
atggcgatgagcccaggtcctttgttcttggtcttcctgttgggtctggttgtgatccctcccactctgtctcaggatgactccaggtacacaaaattcctgactcagcactatgatgccaagccaaaaggccgggacgacagatactgcgaaagtatgatggtgaaaagaaagctaacctctttctgcaaagatgtcaacacctttatccatgacaccaagaacaacatcaaggccatctgtggaaagaaaggaagcccttatggacgaaatttaagaataagcaagtctcgcttccaggtcaccacttgcacacacaaaggaaggtctccccggcctccatgcaggtaccgagcctctaaagggttcagatatattattattggctgtgagaatggctggcctgtccactttgatgagtcttttatcagtccatag

SEQ ID NO: 52 - protein, 145 aa, Artificial Sequence
  Description of Artificial Sequence Consensus sequence
MXMSPGPLXLVFXLGLVVIPPTLAQDXXRYXKFLTQHYDAKPKGRDDRYCESMMKXRKLTSPCKDVNTFIHXTKXNIKAICGXXGSPYGXNXRISXSXFQVTTCTHXGGSPRPPCXYRAXKXFRYIVIACEXGWPVHFDESFISP

SEQ ID NO: 53 - protein, 123 aa, Artificial Sequence
  Description of Artificial Sequence Consensus sequence
QDNXRYXKFLTQHYDAKPXGRDDRYCESMMKXRXLTSPCKDVNTFIHGXKXNIKAICGXXNGXPYXENXRISXSXFQVTTCXHXGGSPXPPCQYRAXXXFRXXVIACEXGXPVHFDESFXSXX

SEQ ID NO: 54 - DNA, 705 bp, Artificial Sequence
  Description of Artificial Sequence Consensus sequence
agcttnacaccgnaggaccctgtctccaggagcacnnagctagacnncntccccngttggaggaaagctggccagctttggaatcnctgttggaagagatgnnnatgagcccangtnctttgttgttggtcttnntgctgngtctggntgtgatncctccnactctggctcagnatnannnnaggtacnnaaaattcctnnntcagcactatgatgccaagccaannggccgggannacagatactgngaaagtatgatgaagnaaagaaagctaacctcgccttgcaaagangtcaacacctttatncatgncaccaagaanaacatcanggccatctgtgganagaanggaagnccttatggagnaaacttnagaataagcaattctcnnttccagntcaccacttgnacgcacnnangagggtctcccnggcctccatgcnngtacnnngcctttaaagatttcaganatattgttattgcctgtgaagatggctggcctgtccacttcgatgagtcttttatcagtccgtagacagcaggcccctggcacagacctaggtctgttttctttttatctcccctcacagccatgatcactggttcancnttcactgtcannggccagaaaatgaattatctgaaatatacttctcctnatttataatgcacagaaataaagatatctcaaaanccanaaaaaa

SEQ ID NO: 55 - DNA, 438 bp, Artificial Sequence
  Description of Artificial Sequence Consensus sequence
atggcgatgagcccaggtcctttgttnttggtcttcntgctgggtctggttgtgatccctcccactctggctcaggatgactncaggtacanaaaattcctgactcagcactatgatgccaagccaaanggccgggacgacagatactgngaaagtatgatgaagaaaagaaagctaacctcnccntgcaaagatgtcaacacctttatccatgncaccaagaacaacatcaaggccatctgtgganagaanggaagcccttatggagnaaacttnagaataagcaantctcncttccaggtcaccacttgcacgcacanaggagggtctcccnggcctccatgcnngtaccgagcctntaaagnnttcagatatattgttattgcctgtgannatggctggcctgtccacttcgatgagtcttttatcagtccntag

SEQ ID NO: 56 - protein, 5 aa, Mus sp.
QNERY

SEQ ID NO: 57 - protein, 6 aa, Mus sp.
QDNSRY